(19) United States
(12) Patent Application Publication
(10) Pub. No.: US 2023/0241081 A1
(43) Pub. Date: Aug. 3, 2023

(54) Method for Treating Melanoma

(71) Applicant: The Regents of the University of Colorado, a body corporate, Denver, CO (US)

(72) Inventors: James Lambert, Denver, CO (US); Steven K. Nordeen, Westminster, CO (US); Yiqun G. Shellman, Aurora, CO (US); M. Scott Lucia, Castle Rock, CO (US)

(73) Assignee: The Regents of the University of Colorado, a body corporate, Denver, CO (US)

(21) Appl. No.: 18/012,975

(22) PCT Filed: Jul. 23, 2021
(86) PCT No.: PCT/US2021/042860

§ 371 Date: Dec. 26, 2022

Related U.S. Application Data
(60) Provisional application No. 63/055,564, filed on Jul. 23, 2020.

(51) Int. Cl. A61K 31/592; A61K 45/06; A61P 35/04; C07K 16/28

(52) U.S. Cl. CPC A61K 31/592; A61K 45/06; A61P 35/04; C07K 16/2818; C07K 16/2827

(57) ABSTRACT

The present invention provides a method of treating melanoma, in a subject in need of such a treatment, a therapeutically effective amount of a compound of the formula: I or a pharmaceutically acceptable salt thereof, a solvate thereof, or a combination thereof, wherein X¹ and X² are as defined herein. Methods of the invention can further include administering other oncolytic agents and/or immunotherapy in treating a patient with melanoma.

### CROSS-REFERENCE TO RELATED APPLICATIONS

This application claims the priority benefit of U.S. Provisional Application No. 63/055,564, filed Jul. 23, 2020, which is incorporated herein by reference in its entirety.

### FIELD OF THE INVENTION

The present invention relates generally to a method for treating melanoma. In particular, the present invention provides a method for treating melanoma by administering a subject in need of such a treatment a therapeutically effective amount of a drug comprising a compound of the formula:

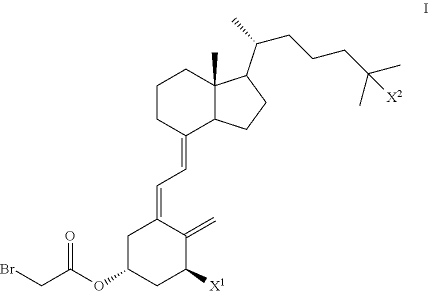

or a pharmaceutically acceptable salt thereof, a solvate thereof, or a combination thereof, wherein X¹ and X² are as defined herein.

### BACKGROUND OF THE INVENTION

The incidence of invasive melanoma cases has increased by 54% in the last decade and is expected to continue to increase rapidly in the United States. Whereas treatments for advanced melanoma have improved significantly in recent years (e.g., targeted therapies against BRAF or MEK, and immunotherapy drugs), not all patients respond, and a significant number of patients experience relapse. For instance, the most promising immune therapy, PD-1 inhibitors and the combination of PD-1+CTLA-4 inhibitors, have 30-40% and 58% initial response rate, respectively, in melanoma, but it is estimated that approximately 25% of melanoma patients will have recurrent disease in less than two years. Thus, there is a critical unmet need for new therapies for melanoma, and particularly for metastatic disease.

In studies of cancer cells and of tumors in mice, vitamin D has been found to have several activities that might slow or prevent the development of cancer, including promoting cellular differentiation, decreasing cancer cell growth, stimulating apoptosis, and reducing angiogenesis. During the study of vitamin D intake on the effects of bone health or other non-cancer outcomes it was discovered that incidence and mortality rate of colorectal cancer, breast cancer, prostate cancer, and pancreatic cancer decreased in subjects with higher intake, or blood levels, of vitamin D. Despite the pre-clinical data and epidemiologic correlations, existing vitamin D analogs have exhibited little or no efficacy in human trials for colorectal, breast, prostate and pancreatic cancer.

### SUMMARY OF THE INVENTION

One aspect of the present invention provides a method for treating melanoma. The method includes administering to a subject in need of such a treatment a therapeutically effective amount of a compound of Formula I:

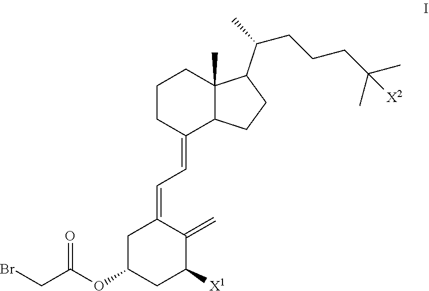

or a pharmaceutically acceptable salt thereof, a solvate thereof, or a combination thereof, wherein
X¹ is H, —OH, or —OC(═O)CH₂BR; and
X² is H or —OH.

In some embodiments, said melanoma is selected from the group consisting of acral melanoma, mucosal melanoma, uveal melanoma, and cutaneous melanoma.

Still in other embodiments, X¹ is —OH. Yet in other embodiments, X¹ is —H. In other embodiments, X¹ is —OC(═O)CH₂Br. In further embodiments, X² is —OH. In other embodiments, X² is H.

In some embodiments, X¹ is —OH and X² is —OH (i.e., compound A). Still in other embodiments, X¹ is —H and X² is —OH. In other embodiments, X¹is —OC(═O)CH₂Br and X² is H. Yet in other embodiments, X¹ is —OC(═O)CH₂Br and X² is —OH.

Another aspect of the invention provides a method for treating melanoma to a subject in need of such a treatment. The method includes administering to the subject a composition comprising a pharmaceutically acceptable excipient and a therapeutically effective amount of a compound of Formula I or a pharmaceutically acceptable salt thereof, a solvate thereof, or a combination thereof. In some embodiments, said composition further comprises an oncolytic agent in an effective amount. Still in other embodiments, said oncolytic agent comprises aldesleukin, binimetinib, encorafenib, cobimetinib, dabrafenib mesylate, dacarbazine, doxorubicin, encorafenib, aldesleukin (IL-2), paclitaxel, talimogene laherparepvec, recombinant interferon α-2b, peginterferon α-2b, ipilimumab, pembrolizumab, trametinib, binimetinib, nivolumab, vemurafenib, tebentafusp, or a combination thereof.

Yet another aspect of the invention provides a method of treating melanoma using combination therapy. The administration of the components can be carried out simultaneously, in tandem, or at different treatment regimens.

Still another aspect of the invention provides a method for treating melanoma by administering to a subject in need of such a treatment a compound of Formula I in combination with an immunotherapy. Such a combination can be carried out simultaneously, in tandem, or at different treatment regimens. In some embodiments, said immunotherapy comprises administering a PD-1 or PD-L1 inhibitor.

### BRIEF DESCRIPTION OF THE DRAWINGS

FIG. 1 shows the effect of compound of compound A (100 nM, square) on growth of cutaneous (LOX-IMVI, MALME-3 M), acral (MB2204) and nodular (MB3961) melanoma cell lines compared to control (circle) determined by real-time kinetic monitoring of cellular proliferation. * Signifies p-values <0.0001 as determined by Student's t-test.

FIG. 2 is a bar graph showing that compound A inhibits the activity of the oncogenic phosphatase, PRL-3 in vitro. In particular, data show the effect of increasing concentrations of compound A on the ability of purified PRL-3 to convert p-nitrophenyl phosphate to p-nitrophenol as measured at 405 nm. * Signifies p-values<0.0001 as determined by Student's t-test.

FIG. 3 shows the growth inhibition of B16 mouse melanoma cells in in vitro culture by treatment with 100 nM compound A.

FIG. 4 shows the growth inhibition in vivo of the B16 mouse melanoma cell line by compound A.

FIG. 5 shows the compound A mediated-inhibition of cell proliferation of cutaneous, acral, and nodular melanoma cell lines with known oncogenic driver mutations. The growth of the indicated melanoma cell lines to ethanol vehicle (circle) or 100 nM compound A (square) was determined by real-time kinetic monitoring of cellular proliferation.

FIG. 6 shows that, in addition to the direct inhibition of PRL-3 activity by the compound A, a second mechanism contributes to compound A-mediated inhibition of PRL-3 activity. FIG. 6 demonstrates that treatment of BT-20 cells with 100 nM compound A induces PRL-3 degradation through a proteasomal-dependent mechanism as evidenced by the ability of the proteasomal inhibitor MG-132 to block compound A-mediated degradation of PRL-3.

### DETAILED DESCRIPTION OF THE INVENTION

Some aspects of the present invention are based on surprising and unexpected discovery by the present inventors that a compound of Formula I or a pharmaceutically acceptable salt thereof, a solvate thereof, a hydrate thereof, or a combination thereof can be used in treating melanoma.

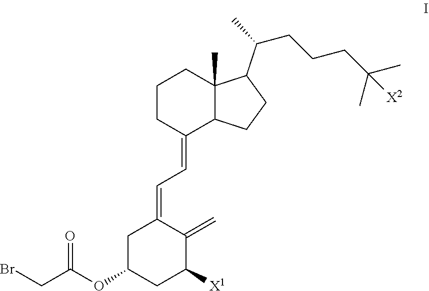

where X¹ is H, —OH, or —OC(═O)CH²Br and X² is H or —OH.

A genome-wide, functional genomic screen revealed that the phosphatase, PRL-3 (phosphatase of the regenerating liver, gene name PTP4A3) was a major target of compound of Formula I and subsequent evaluation demonstrated that PRL-3 expression was required for induction of apoptosis and growth inhibition of some cancer cells by compound of Formula I. Moreover, when a range of cancer cell models was screened for growth inhibition by compound of Formula I, melanoma cell lines were consistently highly sensitive to induction of apoptosis or inhibition of cell growth by compound of Formula I.

Discovery by the present inventors that PRL-3 is a target for compound A was surprising and unexpected. Phosphatases are commonly viewed as off switches that inhibit oncogenic kinases that drive many cancers. PRL-3 is a notable exception. Its involvement in cancer was originally identified as a gene overexpressed in colorectal cancer metastases. Subsequent studies by others have shown that PRL-3 expression is associated with aggressive, metastatic cancers. Investigations have shown that knock down or knock out of PRL-3 reduces or abolishes properties associated with aggressive, metastatic disease in vitro and is required for metastasis in vivo.

PRL-3 belongs to the family of protein tyrosine phosphatases (PTPs). PTPs are important for regulating phosphorylation of many crucial signaling molecules involved in cell cycle, proliferation, and differentiation. Overexpression of PRL-3 has been linked to metastasis and poor prognosis in colorectal, breast, lung, ovarian, hepatocellular cancer, and melanoma. Thus, overexpression of PRL-3 has been found to be associated with metastasis and may be used as a potential biomarker for assessing tumor aggressiveness. However, a consensus on the prognostic value of PRL-3 expression related to its clinical significance has not been reached.

Although high expression of PRL-3 has been linked to increased metastatic behavior of B16F melanoma cells compared to the parental B16 cells, no studies to date have shown any vitamin D analogs having any modulating effect on PRL-3. Thus, PRL-3 is a most intriguing target given the tight association of PRL-3 overexpression and aggressive, metastatic disease.

As conventional vitamin D analogs exert their activity through modulation of vitamin D receptor activity, it was highly unexpected and extremely surprising to discover that compound of Formula I exerted its efficacy independent of vitamin D receptor.

Compounds of the present invention can be readily prepared by one skilled in the art having read the present disclosure as well as experiments disclosed in commonly assigned U.S. Pat. No. 9,539,231, issued Jan. 10, 2017, which is incorporated herein by reference in its entirety.

Pharmaceutical compositions suitable for use in the present invention include compositions wherein the active ingredients are contained in a therapeutically effective amount to achieve its intended purpose. A therapeutically effective amount means an amount effective to prevent development of or alleviate the existing symptoms and underlying pathology of the subject being treated. Determination of the therapeutically effective amount is well within the capability of those skilled in the art.

“Pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Treating” or “treatment” of a disease includes (1) inhibiting the disease, i.e., arresting or reducing the development of the disease or its clinical symptoms; or (2) relieving the disease, i.e., causing regression of the disease or its clinical symptoms.

It is appreciated that the actual preferred amount of each active component in a specific case will vary according to the efficacy of the specific component employed, the particular compositions formulated, the mode of application, and the particular site and organism being treated. For example, the specific dose for a particular patient depends on age, sex, body weight, general state of health, diet, the timing and mode of administration, the rate of excretion, and medicaments used in combination and the severity of the disorder. Dosages for a given host can be determined using conventional considerations, e.g., by customary comparison of the differential activities of the subject compounds and of a known agent, such as by means of an appropriate conventional pharmacological protocol. The effective amount of each active component in the combination may be lower than the effective amount of each active component when it is administered alone.

The pharmaceutical compositions for use in accordance with the present invention can be manufactured in a manner that is known, for example, by means of conventional mixing, dissolving, granulating, levigating, emulsifying, encapsulating, entrapping or lyophilizing processes. In addition, the pharmaceutical compositions can be formulated in conventional manner using one or more physiologically acceptable carriers comprising excipients and auxiliaries, which facilitate processing of the active compounds into preparations that can be used pharmaceutically. Proper formulation is dependent upon the route of administration chosen.

The active components identified in the present invention can be administered to a patient by itself or in pharmaceutical compositions where it is mixed with suitable carriers or excipients at an effective amount. Techniques for formulation and administration of the compounds of the instant invention are well within the capability of those skilled in the art.

Suitable routes of administration in the present invention include oral, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one can administer the composition in a local rather than systemic manner, such as via injection of the composition directly into an affected area, often in a depot or sustained release formulation.

Furthermore, one can administer the composition in a targeted drug delivery system, such as in a liposome coated with an antibody specific for affected cells. The liposomes will be targeted to and taken up selectively by the cells.

The amount of the composition administered depends on the subject being treated and on the subject's weight, the severity of the affliction, the manner of administration and the judgment of the prescribing physician.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinyl-pyrrolidone (PVP). If desired, disintegrating agents can be added, such as the cross-linked polyvinyl pyrrolidone, agar, or alginic acid or a salt thereof such as sodium alginate.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

For injection, the agents of the invention can be formulated in aqueous solutions, preferably in physiologically compatible buffers such as Hank's solution, Ringer's solution, or physiological saline buffer. For transmucosal administration, penetrants appropriate to the barriers to be permeated are used in the formulation. Such penetrants are generally known in the art.

The compositions can be formulated for parenteral administration by injection, e.g., by bolus injection or continuous infusion. Formulations for injection can be presented in unit dosage for, e.g., in ampoules or in multidose containers, with added preservatives. The compositions can take such forms as suspensions, solutions or emulsions in oily or aqueous vehicles, and can contain formulatory agents such as suspending, stabilizing and/or dispersing agents.

Another embodiment of the present invention is directed to the treatment of melanoma by using combination therapy. A patient is administered a combination of an effective amount of the compound of Formula I and at least one of the other known oncolytic agents, such as pembrolizumab. A non-limiting list of other oncolytic agents that may be used in combination with the compound of Formula I for the treatment of melanoma include aldesleukin, binimetinib, encorafenib, cobimetinib, dabrafenib mesylate, dacarbazine, doxorubicin, encorafenib, aldesleukin (IL-2), paclitaxel, talimogene laherparepvec, recombinant interferon alfa-2b, peginterferon alfa-2b, ipilimumab, pembrolizumab, trametinib, binimetinib, nivolumab, tebentafusp, and vemurafenib.

The combination may include antitumor drugs such as cycle-active agents and non-cycle-active agents. Cycle-active agents are drugs that require a cell to be in cycle, i.e., actively going through the cell cycle preparatory to cell division to be cytotoxic. Some of these drugs are effective primarily against cells in one of the phases of the cell. The importance of this designation is that cell cycle-active agents are usually schedule-dependent, and that duration of exposure is as important as and usually more important than dose. In contrast, non-cycle-active agents are usually not schedule-dependent, and effects depend on the total dose administered, regardless of the schedule. Alkylating agents are generally considered to be non-cycle-active, whereas antimetabolites are prototypes of cycle-active compounds.

Immunotherapeutics generally rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, melanoma cells must bear some marker that is amenable to targeting, i.e., is not present on the majority of other cells. Any melanoma markers known to one skilled in the art may be suitable for targeting in the context of the present disclosure. An alternative aspect of immunotherapy is to combine a compound of Formula I with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand.

Examples of immunotherapies currently under investigation or in use are immune adjuvants, cytokine therapy, e.g., interferons α, β, and γIL-1, GM-CSF and TNF, gene therapy, e.g., TNF, IL-1, IL-2, p53, and monoclonal antibodies.

Immunotherapy may be an active immunotherapy or an adoptive immunotherapy. An active immunotherapy typically involves administering an antigenic peptide, polypeptide or protein, or an autologous or allogenic melanoma cell composition or “vaccine”. In adoptive immunotherapy, the patient's circulating lymphocytes, or melanoma infiltrated lymphocytes, are isolated in vitro, activated by lymphokines or transduced with genes for tumor necrosis, and readministered.

Additional objects, advantages, and novel features of this invention will become apparent to those skilled in the art upon examination of the following examples thereof, which are not intended to be limiting.

### EXAMPLES

### Example 1

### NCI-60 Screening Identifies Melanoma as a Target of Compound A:

The Developmental Therapeutics Program at the National Cancer Institute offers a service called the NCI-60 Human Tumor Cell Lines Screen. This program is designed to screen approximately 3,000 small molecules per year for their potential as anticancer agents. The screen utilizes 60 different human tumor cell lines representing leukemia, colon, breast, lung, brain, ovary, prostate, kidney, and melanoma. Importantly, the mutational profile of 24 known cancer genes has been determined for all of the cell lines in the NCI-60 panel allowing for the determination of any potential correlation to drug response and driver mutations. Requests for screening are reviewed by a scientific advisory board and only those conforming to defined guidelines are selected for screening. Compounds selected for screening are initially tested at a single high dose (10⁻⁵ M) against the entire 60 cell line panel.

Compounds that achieve a pre-determined threshold of growth inhibition advance to a 5-dose screen against the panel of cell lines. compound A was submitted and progressed through the 1-dose screen and two rounds of the 5-dose screen. A striking finding resulting from the compound A screening process was that the drug was remarkably effective in killing melanoma cells lines. The melanoma cell lines tested are shown in Table 1.

The LOX-IMVI and MALME-3 M cell lines were selected for confirmatory testing using the IncuCyte Live-Cell kinetic imaging system (Sartorius). As shown in FIG. 1, both cell lines were strongly inhibited by compound A at a low dose of the drug (100 nM).

A critical resource available to this project is the University of Colorado Melanoma Biorepository (UCMB). The UCMB maintains a collection of human melanoma specimens collected at the University of Colorado Hospital and Veterans Administration Hospital. These samples include tumor tissue, over 100 PDX mouse models, cell lines, DNA, and RNA from peripheral blood. Specimens are available on request following review of the UCMB scientific advisory board.

TABLE 1
Melanoma cells lines for testing compound of Formula I

| Cell Line | Genotype |
|---|---|
| LOX-IMVI | BRAF V600E^{-/+} |
| MALME-3M | BRAF V600E^{-/+}, CDKN2A deletion |
| SK-MEL-5 | BRAF V600E^{-/+}, CDKN2A deletion |
| M14 | BRAF V600E^{-/+}, CDKN2A deletion, TP53^{-/+} |
| UACC-62 | BRAF V600E^{-/-}, PTEN^{-/-} |
| MDA-MB-435 | BRAF V600E^{-/+}, CDKN2A deletion, TP53^{-/+} |
| SK-MEL-28 | BRAF V600E^{-/-}, CDK4 R24C^{-/+}, EGFR P753S^{-/-}, |
| | PTEN T167A, TP53 L145R−/− |
| UACC-257 | BRAF V600E^{-/+}, CDKN2A deletion |
| SK-MEL-2 | NRAS Q61R^{-/-}, TP53 G245S^{-/+} |

To test the ability of compound A to inhibit the growth of rare melanomas, an acral cell line (MB2204-Pan-negative) and a nodular cell line (MB3961-NRAS Q61K −/−) were obtained from the UCMB. The MB2204 cell line was chosen because the acral subtype is considered the rarest form of melanoma. Although nodular melanoma is not considered “rare” by the CDMRP guidelines, this line was selected as nodular melanoma represents the most aggressive form of melanoma and is more common in men. As shown in FIG. 1, both the acral and nodular cell lines were significantly growth inhibited by compound A.

### Example 2

### Compound A Binds the PRL-3 Active Site in Silico and Impairs its Catalytic Activity In Vitro:

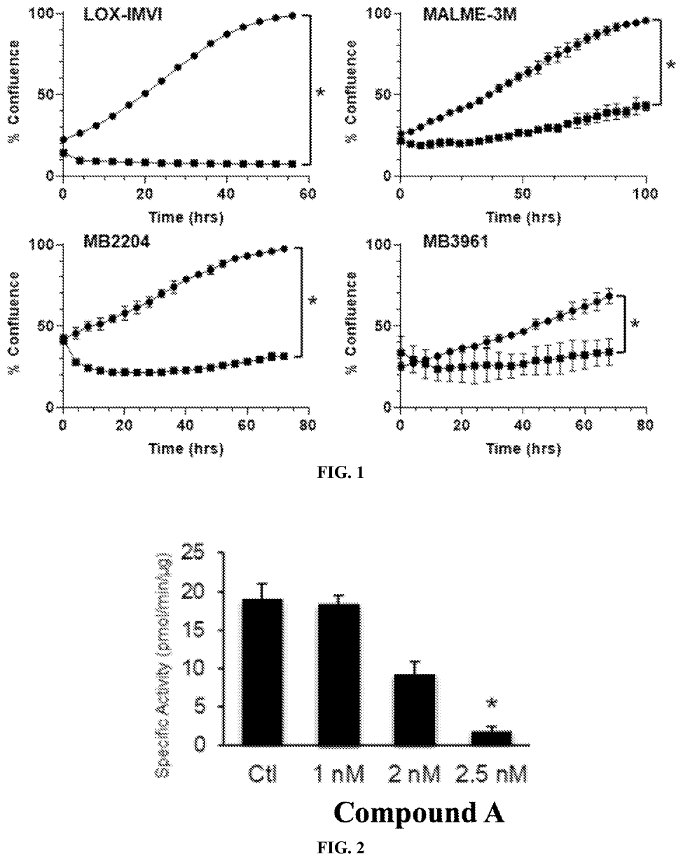

To query the potential of PRL-3 as a target of compound A, in silico docking experiments were carried out using a previously solved NMR structure of PRL-3 and a 3-D model of compound A. The docking studies predicted that the extended structure of compound A binds the active site in the PRL-3 protein in such a way as to place the alkylating bromoacetyl moiety in close proximity to Cys104, a critical catalytic residue in the active site. To assess the functional implications of the in silico study, compound A was assessed for inhibition of the catalytic activity of PRL-3. In vitro phosphatase assays were performed using purified recombinant PRL-3 and the chromogenic substrate paranitrophenyl phosphate. Compound A potently impaired the catalytic activity of PRL-3 in a dose-dependent manner (FIG. 2).

### Example 3

### In Vitro Results:

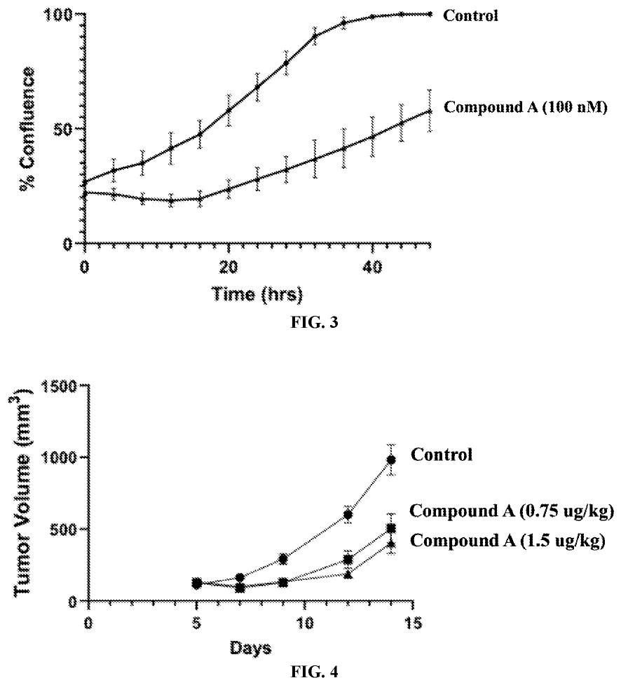

FIG. 3 shows that growth of the aggressive mouse melanoma cell line, B16, is inhibited when treated with compound A at 100 nM. B16 mouse melanoma cells were seeded at 3000 cells per well of a 96 well dish and allowed to attach overnight. The following day the cells were treated with the indicated dose of compound A (triangle) or vehicle control (circle). Cellular proliferation was measured by real-time kinetic imaging.

### Example 4

### In Vivo Results:

FIG. 4 shows the growth inhibition in vivo of the B16 mouse melanoma cell line by compound A. Briefly, B16 mouse melanoma cells were used to establish tumors in c57/B16 mice. After the tumors reached approximately 100 mm³, vehicle control (circle) and the two indicated doses of compound A (square, 0.75 ug/kg and triangle, 1.5 ug/kg) were administered by i.p. injection three times weekly. Tumors were measured on each day of injection by caliper measurement. Compared to untreated controls, a significant reduction in tumor growth was observed.

### Example 5

### Melanoma Cell Line Screening Assay:

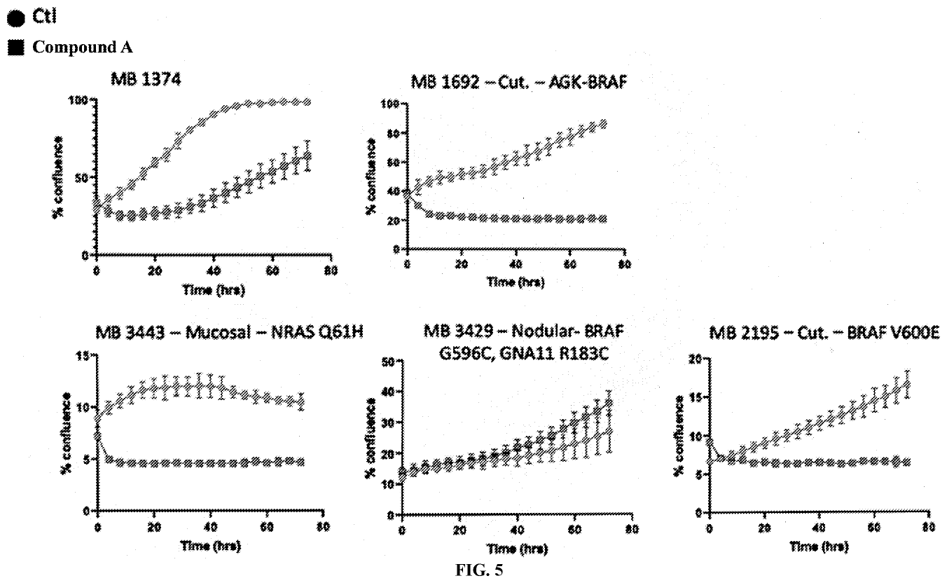

FIG. 5 shows the compound A mediated- inhibition of cell proliferation of cutaneous, acral, and nodular melanoma cell lines with known oncogenic driver mutations. The growth of the indicated melanoma cell lines to ethanol vehicle (circle) or 100 nM compound A (square) was determined by real-time kinetic monitoring of cellular proliferation.

### Example 6

### Inhibition of PRL-3:

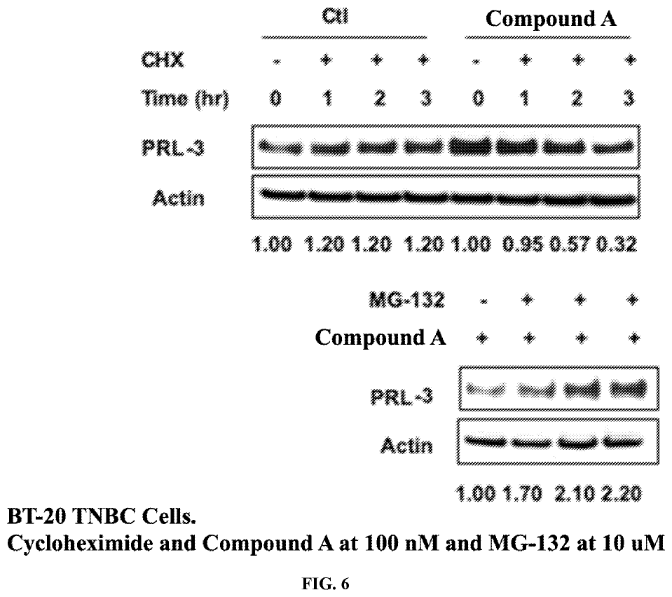

FIG. 6 shows that, in addition to the direct inhibition of PRL-3 activity by the compound A, a second mechanism contributes to compound A-mediated inhibition of PRL-3 activity. FIG. 6 demonstrates that treatment of BT-20 cells with 100 nM compound A induces PRL-3 degradation through a proteasomal-dependent mechanism as evidenced by the ability of the proteasomal inhibitor MG-132 to block compound A-mediated degradation of PRL-3.

The various features and processes described above may be used independently of one another or may be combined in various ways. All possible combinations and subcombinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Throughout this description all ranges described include all values and sub-ranges therein, unless otherwise specified. Additionally, the indefinite article “a” or “an” carries the meaning of “one or more” throughout the description, unless otherwise specified.

Whereas certain example embodiments have been described, these embodiments have been presented by way of example only and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

What is claimed is:

1. A method for treating melanoma to a subject in need of such a treatment, said method comprising administering to the subject a therapeutically effective amount of a compound of the formula: 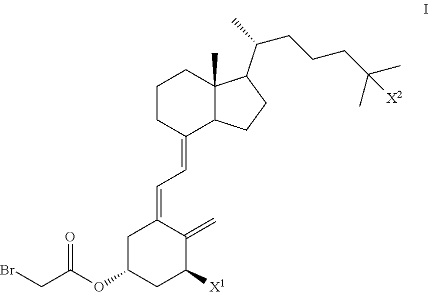
or a pharmaceutically acceptable salt thereof, a solvate thereof, or a combination thereof, wherein
X¹ is H, —OH, or —OC(═O)CH₂BR; and
X² is H or —OH.

2. The method according to claim 1, wherein said melanoma is selected from the group consisting of acral melanoma, mucosal melanoma, uveal melanoma, and cutaneous melanoma.

3. The method according to claim 1, wherein X¹ is —OH.

4. The method according to claim 1, wherein X¹ is —H.

5. The method according to claim 1, wherein X¹ is —OC(═O)CH₂Br.

6. The method according to claim 1, wherein X² is —OH.

7. The method according to claim 1, wherein X² is H.

8. The method according to claim 1, wherein X¹ is —OH and X² is —OH.

9. The method according to claim 1, wherein X¹ is —H and X² is —OH.

10. The method according to claim 1, wherein X¹ is —OC(═O)CH₂Br and X² is —OH.

11. The method according to claim 1, wherein X¹ is —OC(═O)CH₂Br and X² is —H.

12. The method according to claim 1 further comprising the step of administering an oncolytic agent in a therapeutically effective amount.

13. The method according to claim 11, wherein said oncolytic agent comprises aldesleukin, binimetinib, encorafenib, cobimetinib, dabrafenib mesylate, dacarbazine, doxorubicin, encorafenib, aldesleukin (IL-2), paclitaxel, talimogene laherparepvec, recombinant interferon α-2b, peginterferon α-2b, ipilimumab, pembrolizumab, trametinib, binimetinib, nivolumab, vemurafenib, tebentafusp, or a combination thereof.

14. The method according to claim 11, wherein said oncolytic agent is co-administered with said compound of Formula I.

15. The method according to claim 1 further comprising the step of administering an immunotherapy.

16. The method according to claim 14, wherein said immunotherapy comprises administering a PD-1 or PD-L1 inhibitor.
